(19) United States
(12) Patent Application Publication
(10) Pub. No.: US 2018/0271579 A1
(43) Pub. Date: Sep. 27, 2018

(54) MICRO-COLD ATMOSPHERIC PLASMA DEVICE

(71) Applicants: Michael Keidar, Washington, DC (US); Zitong Chen, Ashburn, VA (US); Jonathan Sherman, Potomac, MD (US); Colin N. Young, Alexandria, VA (US); Hayk Simonyan, Washington, DC (US); Jerome Canady, Lakeland, FL (US)

(72) Inventors: Michael Keidar, Washington, DC (US); Zitong Chen, Ashburn, VA (US); Jonathan Sherman, Potomac, MD (US); Colin N. Young, Alexandria, VA (US); Hayk Simonyan, Washington, DC (US); Jerome Canady, Lakeland, FL (US)

(21) Appl. No.: 15/936,290

(22) Filed: Mar. 26, 2018

Related U.S. Application Data
(60) Provisional application No. 62/476,261, filed on Mar. 24, 2017.
(60) Provisional application No. 62/609,629, filed on Dec. 22, 2017.

(51) Int. Cl. A61B 18/04

(52) U.S. Cl. CPC A61B 18/042; A61B 2018/00577

(57) ABSTRACT

A micro-sized CAP (μCAP) employed to target glioblastoma tumors in the murine brain. Various plasma diagnostic techniques were applied to evaluate the physics of helium μCAP such as electron density, discharge voltage, and optical emission spectroscopy. The direct and indirect effects of μCAP on glioblastoma (U87MG-RedFluc) cancer cells in vitro were investigated and indicate that μCAP-generated short- and long-lived species and radicals [i.e. hydroxyl radical (OH), hydrogen peroxide (H2O2), nitrite (NO2-), et al] with increasing tumor cell death in a dose-dependent fashion. Translation of these findings to an in vivo setting demonstrates that intracranial μCAP is effective at preventing glioblastoma tumor growth in the mouse brain. The μCAP device can be safely used in mice, resulting in suppression of tumor growth. These initial observations establish the μCAP device as a potentially useful ablative therapy tool in the treatment of glioblastoma.

### CROSS-REFERENCE TO RELATED APPLICATIONS

The present application claims the benefit of the filing date of U.S. Provisional Patent Application Ser. No. 62/476,261 filed on Mar. 24, 2017 and U.S. Provisional Patent Application Ser. No. 62/609,629 filed on Dec. 22, 2017.

The aforementioned provisional patent applications are hereby incorporated by reference in their entirety.

### STATEMENT REGARDING FEDERALLY SPONSORED RESEARCH OR DEVELOPMENT

This invention was made with government support under Grant No. 1465061 awarded by the National Science Foundation. The government has certain rights in the invention.

### BACKGROUND OF THE INVENTION

### Field of the Invention

The present invention relates to cold atmospheric plasma electrosurgery and more particularly to cold atmospheric plasma devices for use in treatment of glioblastoma.

### Brief Description of the Related Art

Plasma is an ionized gas consisting of ions, electrons, neutral atoms, radicals, and ultraviolet radiation. See, Attri, P.; Kumar, N.; Park, J. H.; Yadav, D. K.; Choi, S.; Uhm, H. S.; Kim, I. T.; Choi, E. H.; Lee, W., “Influence of reactive species on the modification of biomolecules generated from the soft plasma,” Scientific reports 2015, 5, 08221; Ratovitski, E. A.; Cheng, X.; Yan, D.; Sherman, J. H.; Canady, J.; Trink, B.; Keidar, M., “Anti-cancer therapies of 21st century: novel approach to treat human cancers using cold atmospheric plasma,” Plasma Processes and Polymers 2014, 11, (12), 1128-1137; Keidar, M.; Beilis, I., “Plasma engineering: applications from aerospace to bio and nanotechnology,” Academic Press: 2013. Atmospheric plasmas at or near ambient temperature have led to a new field of plasma medicine, (Flynn, P. B.; Busetti, A.; Wielogorska, E.; Chevallier, O. P.; Elliott, C. T.; Laverty, G.; Gorman, S. P.; Graham, W. G.; Gilmore, B. F., “Non-thermal Plasma Exposure Rapidly Attenuates Bacterial AHL-Dependent Quorum Sensing and Virulence,” Scientific reports 2016, 6, 26320; Keidar, M.; Walk, R.; Shashurin, A.; Srinivasan, P.; Sandler, A.; Dasgupta, S.; Ravi, R.; Guerrero-Preston, R.; Trink, B., “Cold plasma selectivity and the possibility of a paradigm shift in cancer therapy,” British journal of cancer 2011, 105, (9), 1295-1301) and cold atmospheric plasma (CAP) has received great attention due to their remarkable potential to affect biological processes. Yousfi, M.; Merbahi, N.; Pathak, A.; Eichwald, O., Low-temperature plasmas at atmospheric pressure: toward new pharmaceutical treatments in medicine. Fundamental & clinical pharmacology 2014, 28, (2), 123-135. In this context, the potential of CAP in diverse bio-medical applications has been explored including disinfection, wound treatments, control of inflammation, blood coagulation, cancer therapy, and regenerative medicine. See, Schmidt, A.; Wende, K.; Bekeschus, S.; Bundscherer, L.; Barton, A.; Ottmuller, K.; Weltmann, K. -D.; Masur, K., “Non-thermal plasma treatment is associated with changes in transcriptome of human epithelial skin cells,” Free radical research 2013, 47, (8), 577-592; Choi, J. W.; Kang, S. U.; Kim, Y. E.; Park, J. K.; Yang, S. S.; Kim, Y. S.; Lee, Y. S.; Lee, Y.; Kim, C. -H., “Novel Therapeutic Effects of Non-thermal atmospheric pressure plasma for Muscle Regeneration and Differentiation,” Scientific Reports 2016, 6, 28829; Daeschlein, G.; Napp, M.; Lutze, S.; Arnold, A.; Podewils, S.; Guembel, D.; Ringer, M., “Skin and wound decontamination of multidrug-resistant bacteria by cold atmospheric plasma coagulation,” JDDG: Journal der Deutschen Dermatologischen Gesellschaft 2015, 13, (2), 143-149; Robert, E.; Vandamme, M.; Brune, L.; Lerondel, S.; Le Pape, A.; Sarron, V.; Riès, D.; Darny, T.; Dozias, S.; Collet, G., “Perspectives of endoscopic plasma applications,” Clinical Plasma Medicine 2013, 1, (2), 8-16; Keidar, M., “Plasma for cancer treatment,” Plasma Sources Science and Technology 2015, 24, (3), 033001.

The efficacy of CAP in proposed applications relies on the synergistic action of reactive oxygen species (ROS), reactive nitrogen species (RNS), free radicals, UV photons, charged particles, and electric fields. Volotskova, O.; Hawley, T. S.; Stepp, M. A.; Keidar, M., Targeting the cancer cell cycle by cold atmospheric plasma. Scientific reports 2012, 2, 00636; Kirson, E. D.; Dbaly, V.; Tovaryg, F.; Vymazal, J.; Soustiel, J. F.; Itzhaki, A.; Mordechovich, D.; Steinberg-Shapira, S.; Gurvich, Z.; Schneiderman, R., “Alternating electric fields arrest cell proliferation in animal tumor models and human brain tumors,” Proceedings of the National Academy of Sciences 2007, 104, (24), 10152-10157; Chen, Z.; Lin, L.; Cheng, X.; Gjika, E.; Keidar, M., “Effects of cold atmospheric plasma generated in deionized water in cell cancer therapy,” Plasma Processes and Polymers 2016, 13, 1151-1156; Keidar, M.; Shashurin, A.; Volotskova, O.; Stepp, M. A.; Srinivasan, P.; Sandler, A.; Trink, B., “Cold atmospheric plasma in cancer therapya,” Physics of Plasmas (1994-present) 2013, 20, (5), 057101. A low dose of ROS and RNS was reported to induce cell proliferation as well as cell death, while a high dose ROS/RNS can damage proteins, lipids, DNA, an induce apoptosis. See, Fridman, G.; Friedman, G.; Gutsol, A.; Shekhter, A. B.; Vasilets, V. N.; Fridman, A., Applied plasma medicine. Plasma Processes and Polymers 2008, 5, (6), 503-533; Dikalov, S. I.; Harrison, D. G., Methods for detection of mitochondrial and cellular reactive oxygen species. Antioxidants & redox signaling 2014, 20, (2), 372-382; Leduc, M.; Guay, D.; Coulombe, S.; Leask, R. L., Effects of Non-thermal Plasmas on DNA and Mammalian Cells. Plasma Processes and Polymers 2010, 7, (11), 899-909; Kalghatgi, S.; Friedman, G.; Fridman, A.; Clyne, A. M., Endothelial cell proliferation is enhanced by low dose non-thermal plasma through fibroblast growth factor-2 release. Annals of biomedical engineering 2010, 38, (3), 748-757. Some results also indicated that exposing cancer cells to CAP resulted in the production of free radicals that could cause apoptotic cell death. Many studies of CAP for cancer therapy have indicated that CAP does not harm normal tissue when applied at the appropriate dosages. Shashurin, A.; Keidar, M.; Bronnikov, S.; Jujus, R.; Stepp, M., Living tissue under treatment of cold plasma atmospheric jet. Applied Physics Letters 2008, 93, (18), 181501; Iseki, S.; Nakamura, K.; Hayashi, M.; Tanaka, H.; Kondo, H.; Kajiyama, H.; Kano, H.; Kikkawa, F.; Hori, M., Selective killing of ovarian cancer cells through induction of apoptosis by nonequilibrium atmospheric pressure plasma. Applied Physics Letters 2012, 100, (11), 113702; Gweon, B.; Kim, M.; Bee Kim, D.; Kim, D.; Kim, H.; Jung, H.; H. Shin, J.; Choe, W., Differential responses of human liver cancer and normal cells to atmospheric pressure plasma. Applied Physics Letters 2011, 99, (6), 063701; Zucker, S. N.; Zirnheld, J.; Bagati, A.; DiSanto, T. M.; Des Soye, B.; Wawrzyniak, J. A.; Etemadi, K.; Nikiforov, M.; Berezney, R., Preferential induction of apoptotic cell death in melanoma cells as compared with normal keratinocytes using a non-thermal plasma torch. Cancer biology & therapy 2012, 13, (13), 1299-1306. Taken together, CAP treatment has been introduced as a cost effective, rapid and selective treatment modality for killing cancer cells, which potentially represents an alternative for conventional cancer therapeutic methods.

CAP may be generated by a range of different plasma devices such as a plasma jet, dielectric barrier discharge, corona discharge, and gliding arc. See, Kolb, J. F.; Mohamed, A. -A. H.; Price, R.; Swanson, R.; Bowman, A.; Chiavarini, R.; Stacey, M.; Schoenbach, K., Cold atmospheric pressure air plasma jet for medical applications. Applied Physics Letters 2008, 92, (24), 241501; Kalghatgi, S. U.; Fridman, G.; Cooper, M.; Nagaraj, G.; Peddinghaus, M.; Balasubramanian, M.; Vasilets, V. N.; Gutsol, A. F.; Fridman, A.; Friedman, G., Mechanism of blood coagulation by nonthermal atmospheric pressure dielectric barrier discharge plasma. IEEE Transactions on plasma science 2007, 35, (5), 1559-1566; Bussiahn, R.; Brandenburg, R.; Gerling, T.; Kindel, E.; Lange, H.; Lembke, N.; Weltmann, K. -D.; von Woedtke, T.; Kocher, T., The hairline plasma: an intermittent negative dc-corona discharge at atmospheric pressure for plasma medical applications. Applied Physics Letters 2010, 96, (14), 143701; Mutaf-Yardimci, O.; Saveliev, A. V.; Fridman, A. A.; Kennedy, L. A., Thermal and nonthermal regimes of gliding arc discharge in air flow. Journal of Applied Physics 2000, 87, (4), 1632-1641. CAP generated plasma can be directly applied to skin cancers. However, most cancers occur inside the body and plasma direct irradiation is not practical due to its high voltage, the formation of discharge in the organ, gas delivery and plasma probe volume. Mirpour, S.; Piroozmand, S.; Soleimani, N.; Faharani, N. J.; Ghomi, H.; Eskandari, H. F.; Sharifi, A. M.; Mirpour, S.; Eftekhari, M.; Nikkhah, M., Utilizing the micron sized non-thermal atmospheric pressure plasma inside the animal body for the tumor treatment application. Scientific Reports 2016, 6, 29048. In this regard, glioblastoma is a highly malignant aggressive neoplasm of the primary central nervous system characterized by rapid growth, extensive angiogenesis, and resistance to all current therapies. Thomas, A. A.; Brennan, C. W.; DeAngelis, L. M.; Omuro, A. M., Emerging therapies for glioblastoma. JAMA neurology 2014, 71, (11), 1437-1444; Ostrom, Q. T.; Gittleman, H.; Fulop, J.; Liu, M.; Blanda, R.; Kromer, C.; Wolinsky, Y.; Kruchko, C.; Barnholtz-Sloan, J. S., CBTRUS statistical report: primary brain and central nervous system tumors diagnosed in the United States in 2008-2012. Neuro-oncology 2015, 17, (suppl 4), iv1-iv62. Thomas, A. A.; Brennan, C. W.; DeAngelis, L. M.; Omuro, A. M., Emerging therapies for glioblastoma. JAMA neurology 2014, 71, (11), 1437-1444.

31. Ostrom, Q. T.; Gittleman, H.; Fulop, J.; Liu, M.; Blanda, R.; Kromer, C.; Wolinsky, Y.; Kruchko, C.; Barnholtz-Sloan, J. S., CBTRUS statistical report: primary brain and central nervous system tumors diagnosed in the United States in 2008-2012. Neuro-oncology 2015, 17, (suppl 4), iv1-iv62

The major limitations of glioblastoma tumor treatment and eventual tumor recurrence are: 1) the brain is susceptible to damage with conventional therapies; 2) tumor cells are very resistant to conventional therapies; 3) many drugs cannot cross the blood-brain barrier to act on brain tumors; 4) the brain has a very limited capacity to repair itself.

### SUMMARY OF THE INVENTION

The present invention provides a plasma device with a reduced gas flow rate and size, which can potentially be used in vivo by circumventing the problems glioblastoma tumor treatment. Here, a new micro-sized CAP (μCAP) device employing helium gas was developed, and the effect of μCAP on glioblastoma both in vitro and in vivo was evaluated.

Cold atmospheric plasma (CAP) treatment is a rapidly expanding and emerging technology for cancer treatment. However, direct CAP jet irradiation is limited to the skin or can be invoked as a supplement during surgery as it only causes cell death in the upper three to five cell layers. In addition, the current cannulas from which the plasma emanates are too large for intracranial applications. To enhance efficiency and expand the applicability of the cold plasma method for brain tumors and reduce gas flow rate and size of the plasma jet, a novel micro-sized CAP (μCAP) was developed and employed to target glioblastoma tumors in the murine brain. Various plasma diagnostic techniques were applied to evaluate the physics of helium μCAP such as electron density, discharge voltage, and optical emission spectroscopy. The direct and indirect effects of μCAP on glioblastoma (U87MG-RedFluc) cancer cells in vitro were investigated and indicate that μCAP-generated short- and long-lived species and radicals [i.e. hydroxyl radical (.OH), hydrogen peroxide (H₂O₂), nitrite (NO₂⁻), et al] with increasing tumor cell death in a dose-dependent fashion. Translation of these findings to an in vivo setting demonstrates that intracranial μCAP is effective at preventing glioblastoma tumor growth in the mouse brain. The μCAP device can be safely used in mice, resulting in suppression of tumor growth. These initial observations establish the μCAP device as a potentially useful ablative therapy tool in the treatment of glioblastoma.

In a preferred embodiment, the present invention is a system for treatment of an area having cancerous cells. The system has a cold atmospheric plasma delivery device with a housing having a channel within the housing, said channel having an entry port for connecting said channel to a source of gas and an exit port for gas flowing through said channel, a first electrode within said channel, a second electrode outside of said channel and a capillary tube connected to said exit port. The system further may have a controller coupled to the delivery device and adapted to control a flow rate of gas flowing from a gas source into said channel and to control a size of a plasma jet exiting said capillary tube.

In another embodiment, the present invention is a system for treatment of an area having cancerous cells. The system has a source of inert gas, a source of electrical energy and a cold atmospheric plasma delivery device. The delivery devices has a housing having a channel within said housing connected to said source of inert gas, said channel having an entry port for connecting said channel to a source of gas and an exit port for gas flowing through said channel and said channel having a diameter greater than 1 mm, a first electrode within said channel, said first electrode being connected to said source of electrical energy, wherein electrical energy applied to said first electrode plasmatizes gas within said channel, a second electrode outside of said channel connected to a ground; and a tube connected to said exit port, wherein said tube has a diameter less than 500 μm, and said tube directs plasmatized gas flowing out of said exit port onto a target. The system may further have a controller coupled adapted to control a flow rate of gas flowing from said gas source into said channel and to control a size of a plasma jet exiting said tube. The inert gas may be helium or another inert gas. The target may be cancerous cells or a medium such as DI water, DMEM or both.

In yet another preferred embodiment, the present invention is a method for eradicating cancerous cells in an area. The method comprises generating a cold atmospheric plasma jet directed at the area having cancerous cells via a capillary tube and controlling the flow rate and size of the plasma jet to the cancerous cells.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description, or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1A is a schematic representation of the micro-sized cold atmospheric plasma setup.

FIG. 1B is an illustration of μCAP direct treatment: μCAP was directly applied to cells in DMEM (100 μL).

FIG. 1C is an illustration of μCAP indirect treatment: DI water was treated with μCAP and then applied to cells in DMEM (70 μL DMEM+30 μL treated DI water).

FIG. 1D is an optical emission spectrum detected from the He μCAP using UV-visible-NIR, in the 250-850 nm wavelength range.

FIG. 2A is a schematic of RMS experimental setup.

FIG. 2B is a graph of electron number and discharge voltage of He.

FIGS. 3A and 3B are RNS concentration in He μCAP treated DI water (3A) and DMEM (3B). Student t-test was performed, and the statistical significance compared to μCAP 5 second treatment is indicated as *p<0.05, **p<0.01, ***p<0.001. (n=3).

FIGS. 4A and 4B show relative methylene blue (MB) concentration for assessing the concentration of hydroxyl free radicals in He μCAP treated DI water (4A) and DMEM (4B). Student t-test was performed, and the statistical significance compared to MB without μCAP treatment (0 second) is indicated as *p<0.05, **p<0.01, ***p<0.001. (n=3).

FIGS. 5A and 5B show H₂O₂ concentration in μCAP treated DI water (5A) and DMEM (5B). Student t-test was performed, and the statistical significance compared to μCAP 5 second treatment is indicated as *p<0.05, **p<0.01, ***p<0.001. (n=3).

FIGS. 6A and 6B show cell viability of U87MG after 24 and 48 hours' incubation with He μCAP indirect treatment (6A) and direct treatment (6B) for 0, 5, 10, 30, 60, and 120 seconds. The ratios of surviving cells for each cell line were calculated relative to controls (DMEM). Student t-test was performed, and the statistical significance compared to cells present in DMEM (0 s) is indicated as *p<0.05, **p<0.01, ***p<0.001. (n=3).

FIG. 7A is a photograph of a new μCAP device for plasma delivery through an intracranial endoscopic tube to target glioblastoma tumors in the mouse brain for in vivo targeting of glioblastoma tumor with He μCAP.

FIG. 7B illustrates Representative in vivo bioluminescence images illustrating glioblastoma tumor volume (i.e. light emission or radiance) at baseline and 2 days following He μCAP or vehicle (helium) treatment. Areas of high photon emission are shown in red and low are shown in blue.

### DETAILED DESCRIPTION OF THE PREFERRED EMBODIMENTS

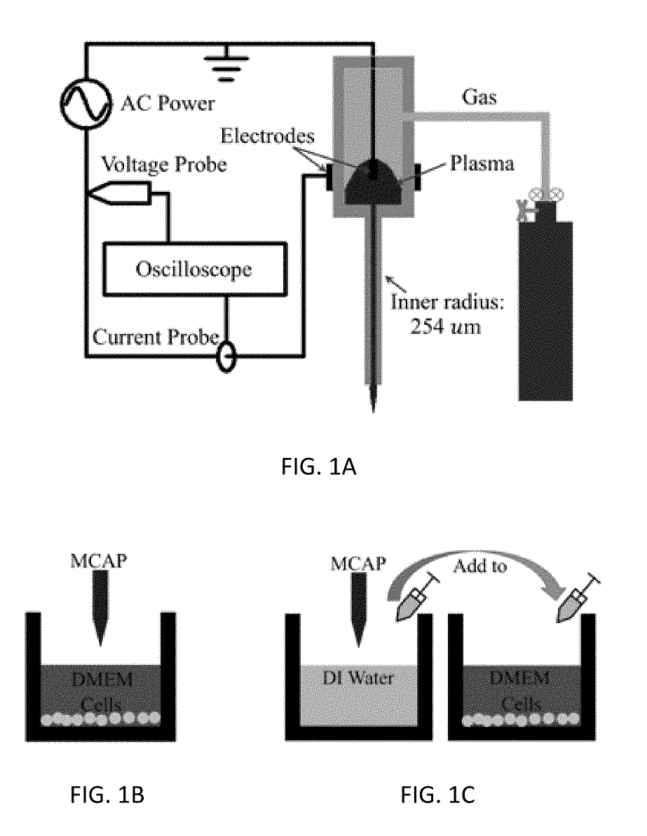

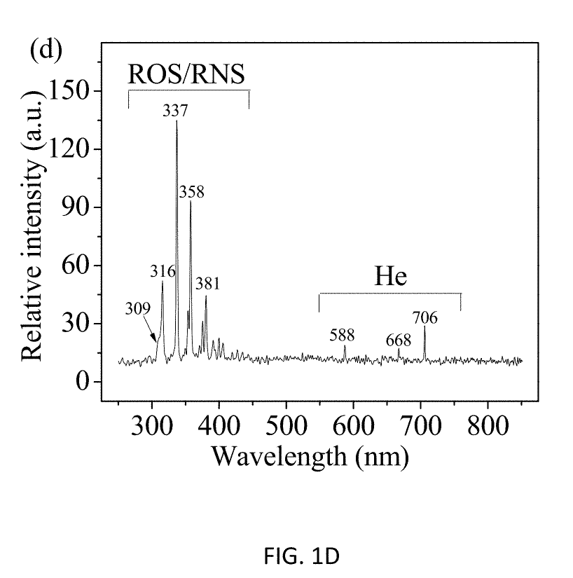

FIG. 1A illustrates the μCAP setup used in this work. At the end of a quartz tube, a 254 μm inner radius capillary tube was attached and insulated by epoxy. The feeding gas for this study was industrial purity He which was injected into the quartz tube with a 0.1 L/min gas flow rate. FIG. 1B illustrates μCAP direct treatment in which the μCAP was used to treat culture medium containing glioblastoma cancer cells. FIG. 1C shows μCAP indirect treatment in which the μCAP was used to first treat DI water. After treatment, the DI water is transferred to culture medium containing cells (70 μL DMEM (containing cells)+30 μL treated DI water, total 100 μL). The time for both direct and indirect treatment was 5, 10, 30, 60, and 120 seconds. FIG. 1D shows the spectrum of the He μCAP with a flow rate of 0.1 L/min. The identification of the emission line and bands was performed mainly according to reference. See, Pearse, R. W. B.; Gaydon, A. G.; Pearse, R. W. B.; Gaydon, A. G., “The identification of molecular spectra,” Chapman and Hall London: 1976; Vol. 29. In the 250-300 nm wavelength range, weak emission bands were detected as NO lines. Species at the wavelength of 316, 337, 358, and 381 nm could be defined as N₂ ³Π or NO β²Π, as both of species have possible optical emission at these wavelengths. The helium bands were assigned between 500 and 750 nm in FIG. 1D. Species at a wavelength of 309 nm could be defines as OH. Both He bands between 250 and 425 nm could be defined as ROS/RNS.

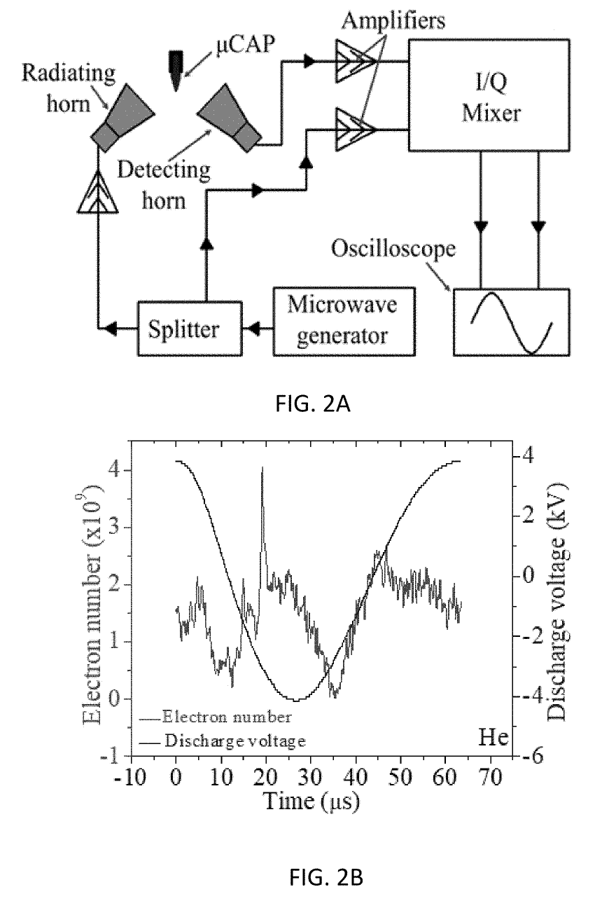

The experimental RMS is shown in FIG. 2A. The dependence of the output RMS signal on parameters of the scattering channel can be expressed as U=AσV, where A is the proportionality coefficient (A=263.8VΩ/cm²) and V is the plasma volume. The volume of the plasma column was determined from Intensified Charged-Coupled Device (ICCD) images. σ is plasma conductivity by using the following expression: σ=2.82×10⁻⁴nₑ+νₘ/(ω²+νₘ²), Ω⁻¹ cm⁻¹, where νₘ is the frequency of the electron-neutral collisions, nₑ is the plasma density, and ω is the angular frequency. See, Shashurin, A.; Shneider, M.; Dogariu, A.; Miles, R.; Keidar, M., Temporary-resolved measurement of electron density in small atmospheric plasmas. Applied Physics Letters 2010, 96, (17), 171502; Lin, L.; Keidar, M., Cold atmospheric plasma jet in an axial DC electric field. Physics of Plasmas (1994-present) 2016, 23, (8), 083529. Combining these equations, yields nₑ=((ω²+νₘ²)U)/((2.82×10⁻⁴Aνₘ)V). Therefore, we can calculate the total electron number in the plasma as Nₑ=nₑV=U(ω²+νₘ²)/(2.82×10⁻⁴Aνₘ). The electron number of He μCAP is presented in FIG. 2B, and the total electron number for one discharge period is 2.4×10⁹.

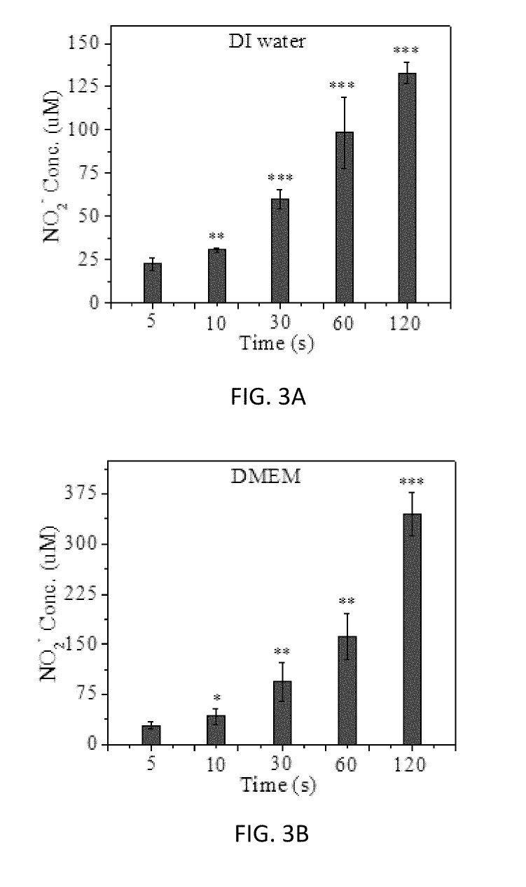

μCAP treatment of DI water and DMEM were performed to induce changes in the concentration of ROS and RNS as a function of the treatment time. Indeed, as shown in FIG. 3, NO₂⁻ concentration in He μCAP treated DI water and DMEM increase with the duration of treatment time. The NO₂⁻ mainly originates as NO (N₂+e→2N+e, N+O₂→+O, 4NO+O₂+2H₂O→4NO₂⁻+4H⁺), while most of NO is formed in the gas phase during the afterglow a few million seconds after the discharge pulse. The NO₂⁻ concentration in DMEM treated with He μCAP is higher than that in DI water.

μCAP treatment of DI (de-ionized) water and DMEM (Dulbecco's Modified Eagle's medium) were performed to induce changes in the concentration of reactive oxygen species (ROS) and reactive nitrogen species (RNS) as a function of the treatment time. Indeed, as shown in FIG. 3, NO₂⁻ concentration in He μCAP treated DI water and DMEM increase with the duration of treatment time. The NO₂⁻ mainly originates as NO (N₂+e→2N+e, N+O₂→NO+O, 4NO+O₂+2H₂O→4NO₂⁻+4H⁺)[36-38], while most of NO is formed in the gas phase during the afterglow a few million seconds after the discharge pulse. The NO₂⁻ concentration in DMEM treated with He μCAP is higher than that in DI water.

MB concentration was used to assess the concentration of .OH. It is known that MB reacts with .OH aqueous solutions leading to a visible color change. Satoh, A. Y.; Trosko, J. E.; Masten, S. J., “Methylene blue dye test for rapid qualitative detection of hydroxyl radicals formed in a Fenton's reaction aqueous solution,” Environmental science & technology 2007, 41, (8), 2881-2887.

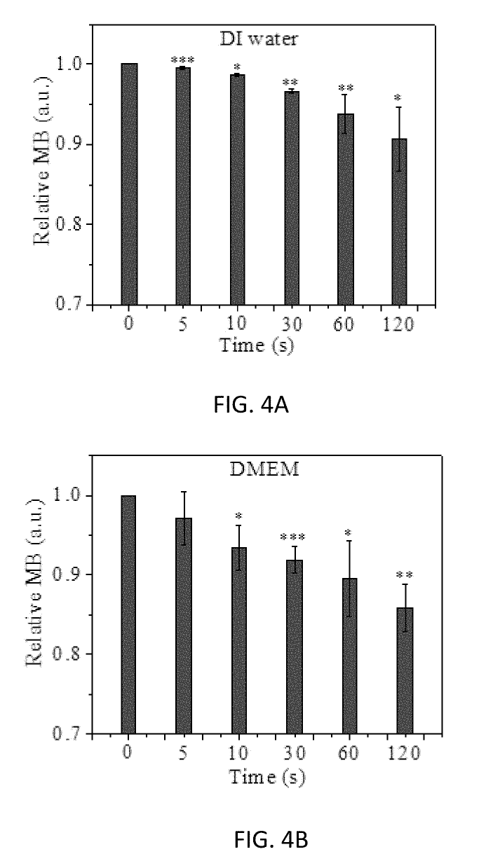

As shown in FIG. 4, the relative MB concentration decreases with treatment time of He μCAP. The relative MB concentration decreases by 9.3% after He μCAP treated DI water for 120 seconds. While this change is large, He μCAP treated DMEM showed a stronger degradation action (14.7%), suggesting that more short-living reactive species are generated in DMEM. Overall, these findings demonstrate that there is an increase in the concentration of OH as a function of μCAP treatment time.

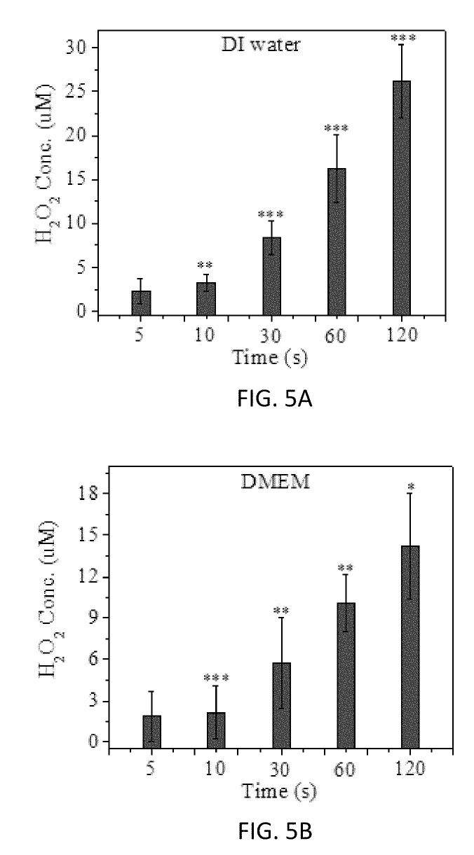

FIG. 5 shows the ROS concentration dependence on treatment time in He μCAP treated DI water and DMEM. H₂O₂ generation might be attributed to the high electron density and energy of the plasma (He→He⁺+e, He⁺+H₂O→H₂O⁺+He, H₂O⁺+H₂O→H₃O⁺+.OH; He+e→He*+e, He*+H₂O→He+.OH+H.; H₂O+e→H₂O*+e, H₂O*→.OH+H.; .OH+OH→H₂O₂). The H₂O₂ concentration increases with the duration of plasma treatment time in both DI water and DMEM, although the concentration of H₂O₂ generated in DI water is higher than DMEM.

The H₂O₂ concentration increases with the duration of plasma treatment time in both DI water and DMEM, although the concentration of H₂O₂ generated in DI water is higher than DMEM.

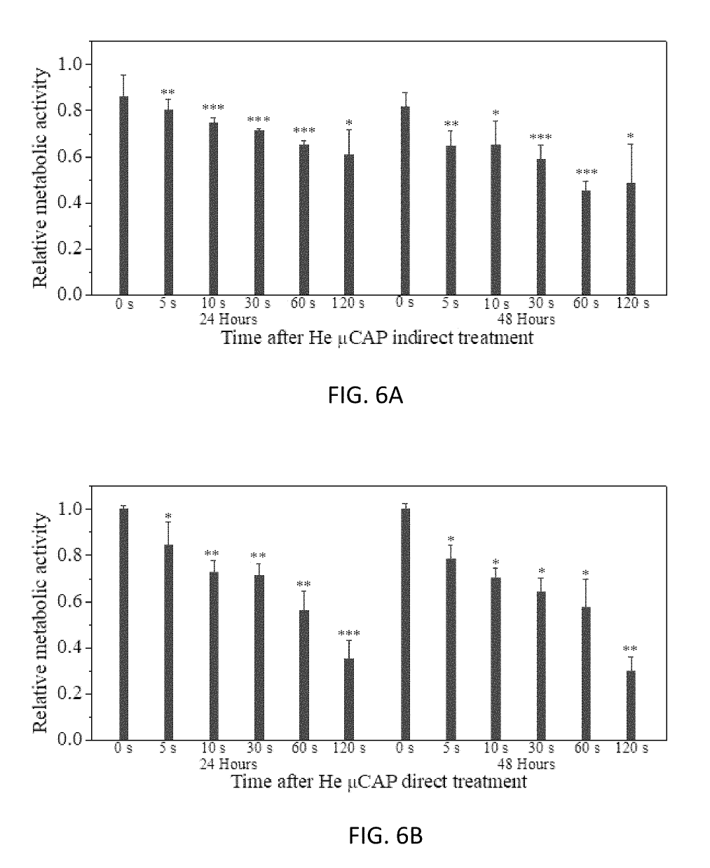

FIG. 6A shows the cell viability of the glioblastoma cancer cells treated with He μCAP indirect treatment after 24 and 48 hours. The cell viability after 24 hours incubation (normalized by DMEM control) decreased by approximately 14.2%, 19.9%, 25.4%, 29.0%, 34.7%, and 39.2%, respectively, according to a 0, 5, 10, 30, 60, and 120 second treatment duration. After 48 hours incubation, the cell viability decreased by approximately 18.3%, 35.6%, 34.8%, 41.1%, 54.7%, and 51.4%, respectively, for a 0, 5, 10, 30, 60, and 120 second treatment duration. FIG. 6B shows the cell viability of glioblastoma cancer cells after 24 and 48 hours incubation with He μCAP direct treatment (treat 100 μL media containing cells) for 0, 5, 10, 30, 60, and 120 seconds duration. The cell viability of the glioblastoma cancer cells after 24 hours incubation (normalized by control) decreased by approximately 15.4%, 27.1%, 28.9%, 44.1%, and 65.1%, respectively, according to 5, 10, 30, 60, and 120 second treatment duration. Similar dose-dependent (i.e. increase treatment time) decreases in cell viability were found with 48 hour incubation.

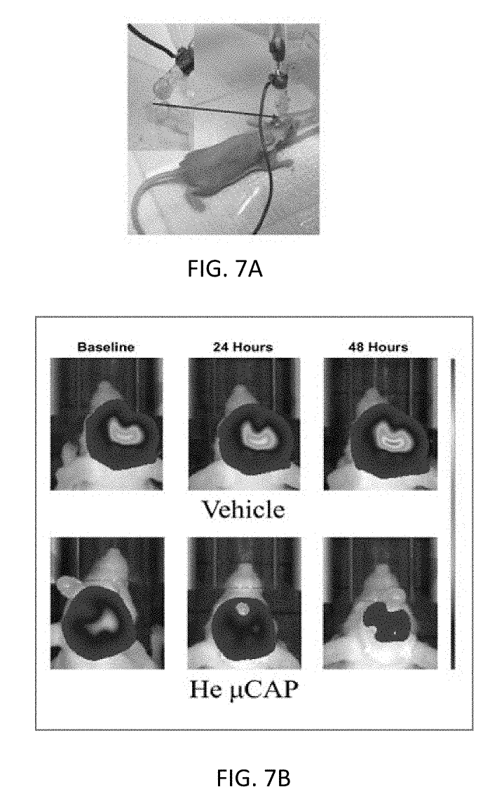

In order to determine the effect of the novel plasma device effect on glioblastoma in vivo, we directly applied He μCAP for 15 seconds to glioblastoma tumors in the brain of living mice via an implanted endoscopic tube as shown in FIG. 7A. Using in vivo bioluminescence imaging (FIG. 7B), the tumor volume in a control animal (helium only) increased nearly 400% over the course of two days, whereas He μCAP treated tumor volume decreased approximately 50% compared with baseline levels. These striking findings demonstrate the potential of μCAP to inhibit glioblastoma tumor growth in vivo.

CAP has received considerable attention for its potential biomedical applications. Emerging fields of application of CAP include wound healing, sterilization of infected tissue, inactivation of microorganisms, tooth bleaching, blood coagulation, skin regeneration, and cancer therapy. This has been collectively termed ‘plasma medicine’. Both direct and indirect applications of CAP have been shown to be effective for treating various cancer cells in vitro and in vivo. However, as discussed above, the treatment of tumors deep within the body has been hampered by the limitations of CAP deliver tools. Thus, we aimed to develop and study how application of a novel He μCAP device could induce high cell death both in vitro and in vivo.

Treating culture media containing cells (direct treatment) was compared to treating DI water and removing it to culture media containing cells (indirect treatment). Our findings suggests that both He μCAP direct/indirect treatment could induce high cell death in glioblastoma cancer cells (FIG. 6), although, in general direct treatment was more effective than that of indirect. Plasma contains the energies ions, free radicals, reactive species, UV radiation, and the transient electric fields inherent with plasma delivery, which interact with the cells and other living organisms. In many cases, it has been reported that plasma induced apoptosis in cancer cells had no adverse effect on the normal cells when administered at the comparable dosage provided to cancer cells. See, Mirpour, S.; Ghomi, H.; Piroozmand, S.; Nikkhah, M.; Tavassoli, S. H.; Azad, S. Z., The selective characterization of nonthermal atmospheric pressure plasma jet on treatment of human breast cancer and normal cells. IEEE Transactions on Plasma Science 2014, 42, (2), 315-322; Yan, D.; Talbot, A.; Nourmohammadi, N.; Cheng, X.; Canady, J.; Sherman, J.; Keidar, M., Principles of using cold atmospheric plasma stimulated media for cancer treatment. Scientific reports 2015, 5, 18339. As previously shown, the inactivation effect on bacteria by UV-radiation in mostly related to DNA/RNA damage at a UV wavelength of 200-280 nm. See., Chen, W.; Huang, J.; Du, N.; Liu, X. -D.; Wang, X. -Q.; Lv, G. -H.; Zhang, G. -P.; Guo, L. -H.; Yang, S. -Z., Treatment of enterococcus faecalis bacteria by a helium atmospheric cold plasma brush with oxygen addition. Journal of Applied Physics 2012, 112, (1), 013304. Thus, it can be argued that UV photons are not the major μCAP species with our experimental setup (FIG. 1). The absorption cross-section of ROS in the UV spectral range is relatively small (Winter, J.; Tresp, H.; Hammer, M.; Iseni, S.; Kupsch, S.; Schmidt-Bleker, A.; Wende, K.; Dünnbier, M.; Masur, K.; Weltmann, K., “Tracking plasma generated H2O2 from gas into liquid phase and revealing its dominant impact on human skin cells,” Journal of Physics D: Applied Physics 2014, 47, (28), 285401), and the species lines between 300-350 are still not clearly determined. Radicals and electrons generated during plasma formation can be either short-lived or long-lived. These radicals or electrons reach a solution and form many complex reactions, which result in the formation of other short- and long-lived radicals or species. Short-lived radicals or species includes superoxide (O₂⁻), nitrite (NO), atomic oxygen (O), ozone (O₃), hydroxyl radical (.OH), singlet delta oxygen (SOD, O₂(¹)), peroxynitrite (ONOO), and etc. See, Graves, D. B., The emerging role of reactive oxygen and nitrogen species in redox biology and some implications for plasma applications to medicine and biology. Journal of Physics D: Applied Physics 2012, 45, (26), 263001; Tian, W.; Kushner, M. J., Atmospheric pressure dielectric barrier discharges interacting with liquid covered tissue. Journal of Physics D: Applied Physics 2014, 47, (16), 165201. Long-lived species include hydrogen peroxide (H₂O₂) and nitrite (NO₂).

In the case of μCAP indirect treatment (treatment of DI water and transfer to cells in culture medium), one can argue that the primary effects are associated with long-lived species (H₂O₂ and NO₂⁻). RNS are known to induce cell death via DNA damage, while ROS can induce cell death by apoptosis and necrosis. S e e, Boehm, D.; Heslin, C.; Cullen, P. J.; Bourke, P., Cytotoxic and mutagenic potential of solutions exposed to cold atmospheric plasma. Scientific reports 2016, 6, 21464; Kim, S. J.; Chung, T., Cold atmospheric plasma jet-generated RONS and their selective effects on normal and carcinoma cells. Scientific reports 2016, 6, 20332. When He is used as a carrier gas, the concentration of H₂O₂ and NO₂⁻ in DI water rises with time (FIG. 3 and FIG. 5). Analyzing cell viability, one can see that He μCAP has a strong effect on glioblastoma (U87MG) cancer cells. The μCAP-generated H₂O₂ and NO₂− concentration increase with treatment time, in line with the linear increase in cancer cell killing efficiency. However, there might be additional antitumor pathways related to H₂O₂ and NO₂⁻. For example, H₂O₂ and NO₂⁻ are believed to generate peroxynitrite (H₂O₂+2NO₂⁻2HONOO⁻) that is known to be toxic to cells. HONOO⁻ is stable at basic pH (DI water <6) or otherwise it immediately decomposes into NO₃⁻. However, HONOO⁻ may be difficult to generate in media because media is buffered and only slightly basic (pH >7). See, Kurake, N.; Tanaka, H.; Ishikawa, K.; Kondo, T.; Sekine, M.; Nakamura, K.; Kajiyama, H.; Kikkawa, F.; Mizuno, M.; Hori, M., Cell survival of glioblastoma grown in medium containing hydrogen peroxide and/or nitrite, or in plasma-activated medium. Archives of biochemistry and biophysics 2016, 605, 102-108. On the other hand, aquaporins (AQPs) play a critical role to facilitate the passive diffusion of H₂O₂, especially AQP8. See, Bienert, G. P.; Chaumont, F., “Aquaporin-facilitated transmembrane diffusion of hydrogen peroxide,” Biochimica et Biophysica Acta (BBA)-General Subjects 2014, 1840, (5), 1596-1604; Ishibashi, K.; Hara, S.; Kondo, S., “Aquaporin water channels in mammals,” Clinical and experimental nephrology 2009, 13, (2), 107-117; Yan, D.; Talbot, A.; Nourmohammadi, N.; Sherman, J. H.; Cheng, X.; Keidar, M., “Toward understanding the selective anticancer capacity of cold atmospheric plasma—model based on aquaporins (Review),” Biointerphases 2015, 10, (4), 040801. Not all AQPs can transport H₂₂, and AQPs show various transportation degrees of H₂O₂ in different types of human tumors. AQP1, 4, 8, and 9 are highly expressed in glioblastoma cell lines. Thus, the distinct expression pattern of AQP8 in glioblastoma might be responsible for cancer cells being sensitive to high H₂O₂ concentrations.

The effect of both short-lived and long-lived species or radicals is plausible when considering μCAP direct treated glioblastoma cancer cells (in vitro, FIG. 6) and tumors in the mouse brain (in vivo, FIG. 7). Of note, DMEM comprises over 30 components such as inorganic salts, amino acids and vitamins and the effect of plasma irradiation on these compounds is still unknown. Similarly, the effect of plasma irradiation in murine brain is also unknown. Therefore, unknown reactive species or radicals could be generated during the irradiation of DMEM and during in vivo application. Short- and long-lived species or radicals in DMEM and mice brain containing .OH, NO, O₂⁻, O, O₃, O₂(¹), ONOO⁻, H₂O₂ and NO₂⁻ are prominent components of antitumor plasma action. The concentration of .OH in DMEM treated by He μCAP increases with treatment time (FIG. 4). .OH derived amino acid peroxides can contribute to cell injury because .OH itself and protein (amino acid) peroxides are able to react with DNA, thereby inducing various forms of damage. S e e, Gebicki, S.; Gebicki, J. M., “Crosslinking of DNA and proteins induced by protein hydroperoxides,” Biochemical Journal 1999, 338, (3), 629-636; Adachi, T.; Tanaka, H.; Nonomura, S.; Hara, H.; Kondo, S. -i.; Hori, M., “Plasma-activated medium induces A549 cell injury via a spiral apoptotic cascade involving the mitochondrialnuclear network,” Free Radical Biology and Medicine 2015, 79, 28-44.

When He μCAP activates DMEM, the concentration of both H₂O₂ and NO₂⁻ rises with treatment time (FIG. 3 and FIG. 5), and synergism of H₂O₂ and NO₂⁻ mentioned above might be an important factor. CAP also produces significant amount of ozone (O₃), which is known to have strongly aggressive effect on cells. See, Fridman, A.; Chirokov, A.; Gutsol, A., Non-thermal atmospheric pressure discharges. Journal of Physics D: Applied Physics 2005, 38, (2), R1-R24. Ozone has a role in the formation of biologically active.

ROS and RNS in aqueous media, which may be responsible for cell death. See, Takamatsu, T.; Uehara, K.; Sasaki, Y.; Miyahara, H.; Matsumura, Y.; Iwasawa, A.; Ito, N.; Azuma, T.; Kohno, M.; Okino, A., Investigation of reactive species using various gas plasmas. RSC Advances 2014, 4, (75), 39901-39905; Kaushik, N.; Uddin, N.; Sim, G. B.; Hong, Y. J.; Baik, K. Y.; Kim, C. H.; Lee, S. J.; Kaushik, N. K.; Choi, E. H., Responses of solid tumor cells in DMEM to reactive oxygen species generated by non-thermal plasma and chemically induced ROS systems. Scientific reports 2015, 5, 08587. Atomic oxygen (O) (including the ground state and all the excited states) is believed to have a significant effect on cells. Superoxide (O₂⁻) generated by plasma can activate mitochondrial-mediated apoptosis by changing mitochondrial membrane potential and simultaneously up-regulates pro-apoptotic genes and down-regulates anti-apoptotic genes for activation of caspases resulting in cell death. Riedl, S. J.; Shi, Y., “Molecular mechanisms of caspase regulation during apoptosis,” Nature reviews Molecular cell biology 2004, 5, (11), 897-907.

Singlet delta oxygen O₂(¹Δg) is another important ROS with the excitation energy of 0.98 eV. Highly reactive molecule O₂(¹Δg) not only produces oxidative damage in many biological targets but is also a primary active species in the selective killing of tumor cells in the emerging cancer therapy. Moreover, nitrite (NO) and superoxide (O₂⁻) can easily form ONOO⁻ once they collide. See, Pacher, P.; Beckman, J. S.; Liaudet, L., Nitric oxide and peroxynitrite in health and disease. Physiological reviews 2007, 87, (1), 315-424. ONOO⁻ is a powerful oxidant and nitrating agent, which is known to be highly damaging to tumor cells. See, Cheng, X.; Sherman, J.; Murphy, W.; Ratovitski, E.; Canady, J.; Keidar, M., “The effect of tuning cold plasma composition on glioblastoma cell viability,” PloS one 2014, 9, (5), e98652.

Overall, the above results and discussion indicate that both direct and indirect routes of delivering CAP might be useful and should be considered in a clinical medical application. A further understanding of the precise underlying mechanisms will allow for determination of the “best” combination when used as a treatment strategy.

The μCAP setup consists of a 2 electrode assembly with a central powered electrode and a grounded outer electrode wrapped around the outside of quartz tube (FIG. 1A). At the end of the quartz tube, a 254 μm inner radius capillary tube was attached and insulated by epoxy. The two electrodes were connected with a high voltage power supply. The feeding gas for this study was industrial purity helium (He) which was injected into the quartz tube with a 0.1 L/min gas flow rate. The voltage and current were measured by an oscilloscope with high voltage and current probes.

UV-visible-NIR, a range of wavelength 200-850 nm, was investigated on plasma to detect various RNS and ROS (nitrogen [N₂], nitric oxide [—NO], nitrogen cation [N⁺²], atomic oxygen [O], and hydroxyl radicals [—OH]). The spectrometer and detection probe were purchased from Stellar Net Inc. The optical probe was placed at a distance of 1.0 cm in front of the plasma jet nozzle. Data were collected with an integration time of 100 ms.

The experimental RMS system is schematically presented in FIG. 2A. Two microwave horns were used for radiation and detection of microwave signal. Microwave radiation linearly polarized was scattered on the collinearly-oriented plasma channel and the scattered signal was then measured. The detection of the scattered signal was accomplished using a homodyne scheme by means of I/Q mixer, providing in-phase (I) and quadrature (Q) outputs. For the entire range of scattered signals, the amplifiers and mixer were operated in linear mode. The total amplitude of the scattered microwave signal was determined by: U=\sqrt{I²+Q²}.

Human glioblastoma cancer cells (U87MG, Perkin Elmer) were cultured in Dulbecco's Modified Eagle Medium (DMEM, Life Technologies) supplemented with 10% (v/v) fetal bovine serum (Atlantic Biologicals) and 1% (v/v) penicillin and streptomycin (Life Technologies). Cultures were maintained at 37° C. in a humidified incubator containing 5% (v/v) CO₂.

A fluorimetric hydrogen peroxide assay Kit (Sigma-Aldrich) was used for measuring the amount of H₂O₂, according to the manufacturer's protocol. Briefly, 50 μl of standard curve, control, and experimental samples were added to 96-well flat-bottom black plates, and then 50 μl of Master Mix was added to each of well. The plates were incubated for 20 min at room temperature protected from light and fluorescence was measured by a Synergy H1 Hybrid Multi-Mode Microplate Reader at Ex/Em: 540/590 nm.

RNS level were determined by using a Griess Reagent System (Promega Corporation) according to the instructions provided by the manufacturer. Briefly, 50 μl of samples and 50 μl of the provided Sulfanilamide Solution were added to 96-well flat-bottom plates and incubated for 5-10 minutes at room temperature. Subsequently, 50 μl of the NED solution was added to each well and incubated at room temperature for 5-10 minutes. The absorbance was measured at 540 nm by Synergy H1 Hybrid Multi-Mode Microplate Reader.

An MB solution was prepared by dissolving MB podwer in DI water and DMEM. MB solutions (100 μl per well, 0.01 g/L) in a 96-well flat-bottom black plate were treated by He μCAP for 5, 10, 30, 60, and 120 seconds. The gap between the outlet of μCAP and the surface of the samples was around 3 mm. As a control, two untreated MB solutions in triplicate were transferred to a 96-well flat-bottom black plate. .OH was measured as the absorbance at 664 nm by a Synergy H1 Hybrid Multi-Mode Microplate Reader.

U87 cells were plated in 96-well flat-bottom microplates at a density of 3000 cells per well in 70 μL of complete culture medium. Cells were incubated for 24 hours to ensure proper cell adherence and stability. On day 2, 30 μL of DI water was treated by He μCAP for 0, 5, 10, 30, 60, and 120 seconds, and was added to cells. Cells were further incubated at 37° C. for 24 and 48 hours. The cell viability of the glioblastoma cancer cells was measured for each incubation time point with an MTT assay. 100 μL of MTT solution (3-(4,5-dimethylthiazol-2-yl)-2,5-diphenyltetrazolium bromide) (Sigma-Aldrich) was added to each well followed by a 3-hour incubation. The MTT solution was discarded and 100 μL per well of MTT solvent (0.4% (v/v) HCl in anhydrous isopropanol) was added to the wells. The absorbance of the purple solution was recorded at 570 nm with a Synergy H1 Hybrid Multi-Mode Microplate Reader.

U87 cells were plated in 96-well flat-bottom microplates at a density of 3000 cells per well in 100 μL of complete culture medium. Cells were incubated for 24 hours to ensure proper cell adherence and stability. On day 2, the cells were treated by He μCAP for 0, 5, 10, 30, 60, and 120 seconds. Cells were further incubated at 37° C. for 24 and 48 hours. An MTT assay was used to assess cell viability as described above.

All animal protocols were approved by the George Washington University Institutional Animal Care and Use Committee. 8 week old female athymic nude mice (Charles River, NU(NCr)-Foxn1^{nm}) were anesthetized intraperitoneally (i.p.) [Ketamine (100 mg/kg) mixed with Xylazine (10 mg/kg)] and placed in a stereotaxic frame. The surface of the skull was visualized with a dissecting microscope and horizontally leveled between bregma and lambda. A small hole was drilled at the desired location. U87MG were resuspended in DMEM at a concentration of 5×10⁵ cell/μL and injected into the frontal lobe at the following coordinates (relative to Bregma) using a Hamilton syringe: 2.2 mm ventral from the dorsal surface of the skull, 1.0 mm caudal, and 2.0 lateral. 5×10⁵ cells were injected at a depth of 2.2 mm and the syringe was then retracted to 1.8 mm and an additional 5×10⁵ cells were then administered. To allow for delivery of μCAP, an endoscopic tube was then implanted at a depth of 2.2 mm and secured in place with dental cement. Mice were allowed to recover for 7 days and He μCAP or vehicle control (He alone) was then administered. In brief, mice were anesthetized with isofluorane and CAP was applied via the implanted endoscopic tube at 5-second intervals, followed by 15 seconds off, for a total CAP treatment time of 15 seconds. Tumor size was estimated using in vivo bioluminescent imaging before and for up to 48 hours post CAP or vehicle treatment. For bioluminescent imaging, animals were anesthetized with isoflurane and the substrate luciferin was administered i.p. (150 mg/kg). The mice were then transferred to the light-sealed imaging cabinet of an IVIS Lumina K machine and positioned in a nose cone in order to maintain anesthesia. Bioluminescent images were acquired using a charge-coupled device camera cooled to −80° C. to achieve maximal sensitivity. Images were acquired at 10 minutes post substrate injection with an exposure time of 20 seconds, medium binning, F/Stop=1, and EM gain off.

Results were plotted using Origin 8 as mean±standard deviation. Student t-test was used to check the statistical significance (*p<0.05, **p<0.01, ***p<0.001).

In summary, the effect of a newly developed μCAP on glioblastoma both in vitro and in vivo has been demonstrated. A variety of diagnostics tools were applied to the μCAP including optical emission spectroscopy, microwave scattering, and potential measurement, which supplied evidence for reactions of plasma-generated species in media and the mouse brain. The μCAP direct treatment has a stronger effect than the indirect treatment due to the synergetic effect of short- and long-lived species, while a strong effect of indirect μCAP treatment on cancer cells is likely attributed to the action of long-lived species. The μCAP is safe for mice and suppresses tumor growth in the mouse brain. These initial observations establish μCAP as a potentially useful ablative therapy in glioblastoma.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A system for treatment of an area having cancerous cells, comprising:
a cold atmospheric plasma delivery device comprising:
a housing having a channel within said housing, said channel having an entry port for connecting said channel to a source of gas and an exit port for gas flowing through said channel;
a first electrode within said channel;
a second electrode outside of said channel; and
a capillary tube connected to said exit port.

2. A system for treatment of an area having cancerous cells according to claim 1, further comprising:
a controller coupled adapted to control a flow rate of gas flowing from a gas source into said channel and to control a size of a plasma jet exiting said capillary tube.

3. A system for treatment of an area having cancerous cells, comprising:
a source of inert gas;
a source of electrical energy;
a cold atmospheric plasma delivery device comprising:
a housing having a channel within said housing connected to said source of inert gas, said channel having an entry port for connecting said channel to a source of gas and an exit port for gas flowing through said channel and said channel having a diameter greater than 1 mm;
a first electrode within said channel, said first electrode being connected to said source of electrical energy, wherein electrical energy applied to said first electrode plasmatizes gas within said channel;
a second electrode outside of said channel; and
a tube connected to said exit port, wherein said tube has a diameter less than 500 μm, and said tube directs plasmatized gas flowing out of said exit port onto a target.

4. A system for treatment of an area having cancerous cells according to claim 3, further comprising:
a controller coupled adapted to control a flow rate of gas flowing from said gas source into said channel and to control a size of a plasma jet exiting said tube.

5. A system for treatment of an area having cancerous cells according to claim 3, wherein said inert gas comprises helium.

6. A system for treatment of an area having cancerous cells according to claim 3, wherein said target comprises cancerous cells.

7. A system for treatment of an area having cancerous cells according to claim 3, wherein said target comprises a medium.

8. A method of eradicating cancerous cells in an area, the method comprising:
generating a cold atmospheric plasma jet directed at the area having cancerous cells via a capillary tube; and controlling the flow rate and size of the plasma jet to the cancerous cells.
